(19) United States
(12) Patent Application Publication
(10) Pub. No.: US 2026/0007540 A1
(43) Pub. Date: Jan. 8, 2026

(54) OSTOMY BARRIER APPLIANCE WITH LOCALIZED ADJUSTABLE CONVEXITY

(71) Applicant: HOLLISTER INCORPORATED, Libertyville, IL (US)

(72) Inventor: Gregory J. CZAPLEWSKI, Bloomingdale, IL (US)

(73) Assignee: HOLLISTER INCORPORATED, Libertyville, IL (US)

(21) Appl. No.: 18/881,184

(22) PCT Filed: Apr. 25, 2023
(86) PCT No.: PCT/US2023/066156

§ 371 Date: Jan. 3, 2025

Related U.S. Application Data
(60) Provisional application No. 63/389,226, filed on Jul. 14, 2022.

(51) Int. Cl. A61F 5/443

(52) U.S. Cl. CPC A61F 5/443

(57) ABSTRACT

A convex ostomy barrier assembly for attaching an ostomy appliance to a peristomal skin surrounding a stoma includes a skin barrier, an inlet opening defined in the skin barrier for receiving the stoma, and a convexity adjusting device. The convexity adjusting device is configured to provide a convexity to the skin barrier. The convexity adjusting device is configured to provide a localized and customized adjustment of at least one characteristic of the convexity.

### BACKGROUND

This disclosure is related to a convex ostomy barrier. More particularly, the present disclosure pertains to an ostomy barrier appliance having a localized adjustable convexity feature.

Ostomy pouches for collecting bodily waste are used by individuals who have had surgery such as a colostomy, ileostomy, or urostomy. An ostomy pouch may be attached to a user via an ostomy barrier, which is configured to seal against peristomal skin surfaces and protect the peristomal surfaces from exposure to stomal effluent. However, the topography of stomas and peristomal surfaces surrounding stomas varies among patients, and sealing an ostomy appliance against such different peristomal surfaces and stomas remain as an area for further improvements. For example, a stoma may protrude more or less, or may even be flush or recessed.

A person with an ostomy having a stoma that is flush or recessed may find that applying external support or pressure from a barrier in the peristomal region aids in directing the discharge of effluent from the stoma directly into the ostomy pouch. Accordingly, the effectiveness of an adhesive seal between the ostomy barrier and the peristomal skin surface (i.e., a seal formed by the adhesive layer) may be prolonged. Thus, convex inserts and convex ostomy barriers, such as ADAPT® convex barrier rings available through the Applicant of the present application, have been developed to apply pressure around such peristomal regions.

However, the convexity of a conventional convex ostomy barrier or insert is fixed and may not work efficiently for all ostomates. Thus, convex ostomy barriers and convex inserts of various convexity depths have been made available in the market. Further, US 2019/0231580, which is assigned to the Applicant of the present application and incorporated herein by reference, discloses ostomy barrier appliances including a convexity adjusting device, which is configured to allow a user to adjust the convexity depth of a skin barrier. However, these ostomy barrier systems are configured to adjust only the convexity depth uniformly about the skin barrier.

Accordingly, it is desirable to provide an improved ostomy barrier appliance that allows localized adjustment of various characteristics of the barrier convexity according to the topography of user's stoma and peristomal surface.

### BRIEF SUMMARY

A convexity adjusting device for an ostomy skin barrier configured to locally adjust a convexity of a skin barrier is provided according to various embodiments.

In one aspect, a convex ostomy barrier assembly for attaching an ostomy appliance to a peristomal skin surrounding a stoma may include a skin barrier, an inlet opening defined in the skin barrier for receiving the stoma, and a convexity adjusting device. The convexity adjusting device may be configured to provide a convexity to the skin barrier. The convexity adjusting device may be configured to provide a localized and customized adjustment of at least one characteristic of the convexity, wherein the at least one characteristic of the convexity includes a compressibility or slope.

In an embodiment, the convexity adjusting device may include a plurality of radially extending members and a plurality of adjusting members. Each of the plurality of radially extending members may be separated from an adjacent radially extending member by a gap. Each of the radially extending members may be configured to be adjusted independently by one of the plurality of adjusting members. Each of the plurality of radially extending members and the corresponding adjusting member may be configured to provide a localized adjustment of a slope, compressibility and/or depth of the convexity. In some embodiments, the plurality of radially extending members may include eight or sixteen radially extending members.

In an embodiment, each of the plurality of adjusting members may be formed by a bi-stable clip configured to adjust the slope, compressibility and/or depth of the convexity of the skin barrier. The bi-stable clip may be configured to be positioned and held in at least two different stable positions, wherein each stable position provides a different slope, compressibility and/or depth of the corresponding radially extending member. The at least two different stable positions may include a first position and a second position, wherein the radially extending member provides a higher compressibility when the corresponding bi-stable clip is set in the first position when compared to the second position.

In an embodiment, the convex adjusting device may be formed by a convex insert comprising a base including a lip defined at an inner periphery, wherein the plurality of radially extending members extends from the base. Each of the radially extending members may include a tab, wherein each of the plurality of adjusting members may be secured in place between the lip and the tab.

In an embodiment, the bi-stable clip may include a first end, a second end, and a hinge arranged between the first and second end. The hinge may be configured to allow the bi-stable clip to bend and change position between the first position and the second position. In such an embodiment, the first end of the bi-stable clip may be attached to an inner surface of the lip of the base and the second end of the bi-stable clip may be arranged against the tab. The hinge may be formed by an indentation or score provide on an inner surface of the bi-stable clip facing the corresponding radially extending member or on an outer surface of the bi-stable clip facing the inlet opening.

In an embodiment, the convex ostomy barrier assembly may be provided with each of the plurality of bi-stable clips arranged in the first position to provide a generally uniform and relatively firm compressibility. The convex ostomy barrier assembly may be configured to allow a user to manipulate one or more of the plurality of bi-stable clips to change the position of the bi-stable clips to the second position to adjust the compressibility at one or more locations.

In an embodiment, the plurality of adjusting members may be attached to an adjusting member base and formed as a single-piece device. In such an embodiment, the adjusting member base may form a first end of each of the plurality of adjusting members and adjacent adjusting members may be connected via the adjusting member base.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a perspective distal side view of an ostomy barrier appliance, including a convexity adjusting device according to an embodiment with a portion removed to illustrate its layered structure.

FIG. 2 is a partial enlarged view of the convexity adjusting device of FIG. 1.

FIG. 3 is a cross-section view of the convexity adjusting device according to an embodiment with an adjusting member in a first position.

FIG. 4 is a cross-section view of the convexity adjusting device of FIG. 3 with the adjusting member in a second position.

FIG. 5 is a perspective distal side view of an ostomy barrier appliance, including a convexity adjusting device according to another embodiment with a portion removed to illustrate its layered structure.

FIG. 6 is a partial enlarged view of the convexity adjusting device of FIG. 5.

FIG. 7 is an illustration of a depth of a convex skin barrier.

FIG. 8 is an illustration of compressibility of a convex skin barrier.

FIG. 9 is an illustration of flexibility of a convex skin barrier.

FIGS. 10A and 10B are illustrations of tension locations of a convex skin barrier.

FIG. 11 is an illustration of a slope of a convex skin barrier.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular.

The characteristics of convexity of a skin barrier can include depth, compressibility, flexibility, tension location, and slope. See, McNichol, L., Cobb, T., Depaifve, Y., Quigley, M., Smitka, K., & Gray, M., Characteristics of Convex Skin Barriers and Clinical Application: Results of an International Consensus Panel, J Wound Ostomy Continence Nurs., (2021) 48(6), 524-532, Abstract. The depth of a convex skin barrier is defined as a distance from the apex of the dome to the base of the skin barrier. Id, at pg. 526. The depth D can be measured as a magnitude of the convexity from the base lying on the peristomal skin to the highest point of the skin barrier as shown in FIG. 7. Id. Individual user's peristomal condition, such as depths of creases and folds around the stoma, should be carefully considered when determining a depth of a convex skin barrier to provide an optimal seal around the peristomal skin. Id.

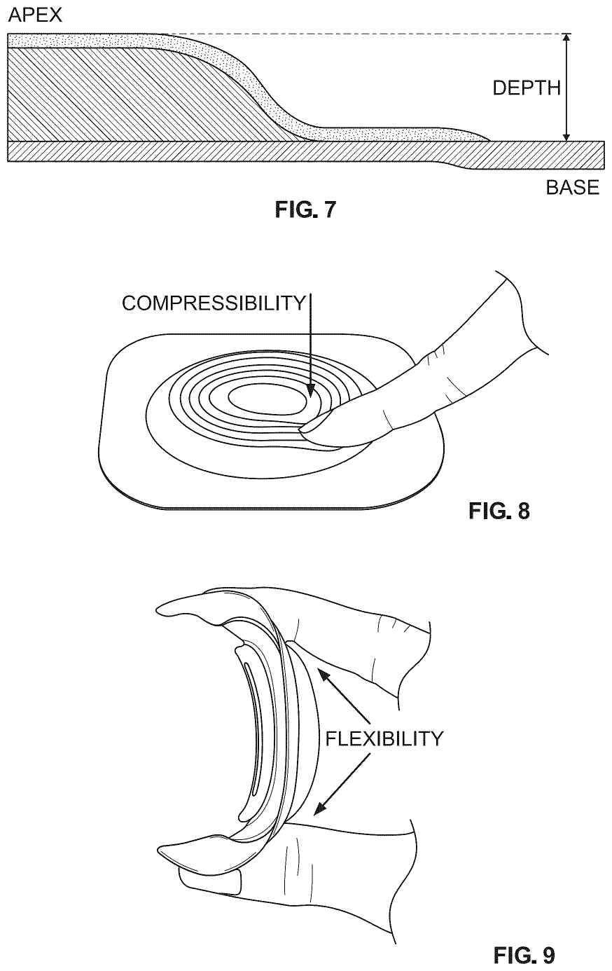

The compressibility of a convex skin barrier is defined as a capacity of the convex dome to be displaced or flattened as illustrated in FIG. 8. Id, at pg. 528. The compressibility may be measured as a force required to displace or flatten the dome portion of a convex skin barrier by a predetermined distance. A relatively easily compressible soft convex barrier may conform better to users with postoperative edema and/or a relatively firm abdominal. Id. A relatively less compressible firm convex barrier may apply more pressure on the peristomal skin to provide support needed for users with a relatively soft abdominal tone and/or creases around the stoma. Id.

The flexibility of a convex skin barrier is defined as how easily the convex skin barrier can bend as illustrated in FIG. 9. Id, at pg. 529. The flexibility is an important characteristic to consider when a skin barrier needs to bend to conform to abdominal contours. Id. A relatively more flexible convex skin barrier may work well for users with multiple creases around stoma due to loose skin. Id.

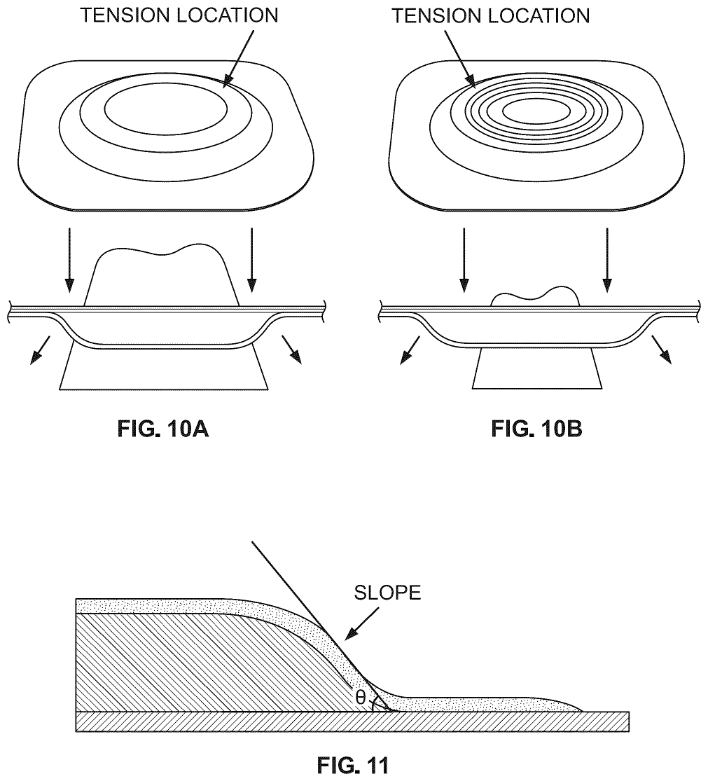

The tension location of a convex skin barrier is defined as the position in which a convex dome exerts downward and outward forces on the peristomal topography as illustrated in FIGS. 10A and 10B. Id, at pg. 530. A convex skin barrier configured to apply a tension close to a stoma may provide a consistent and reliable seal around the stoma that is flush to the skin or retraced below the skin. Id. For users with creases and folds around the stoma, a convex barrier skin barrier configured to apply a tension away from the stoma may help flatten the peristomal skin to provide a good seal. Id.

The slope of a convex skin barrier is defined as an angle from the base of the dome to a periphery of the apex of the dome as illustrated in FIG. 11. Id, at pg. 53. Creases and folds around the stoma can compromise a seal between a skin barrier and the skin. Adjusting the slope of a convex skin barrier according to user's peristomal topography can improve the seal. For example, a convex skin barrier with a relatively small slope and wider plateau may help flattend the peristomal skin creases and folds to achieve a good seal. Id.

Customizing and adjusting the depth, compressibility, flexibility, tension location, and/or slope of a convex skin barrier according to user's peristomal topography can provide an optimal seal around the stoma. The present disclosure provides an ostomy barrier appliance including a convexity adjusting device configured to locally adjust one or more of the convexity characteristics of a skin barrier according to various embodiments.

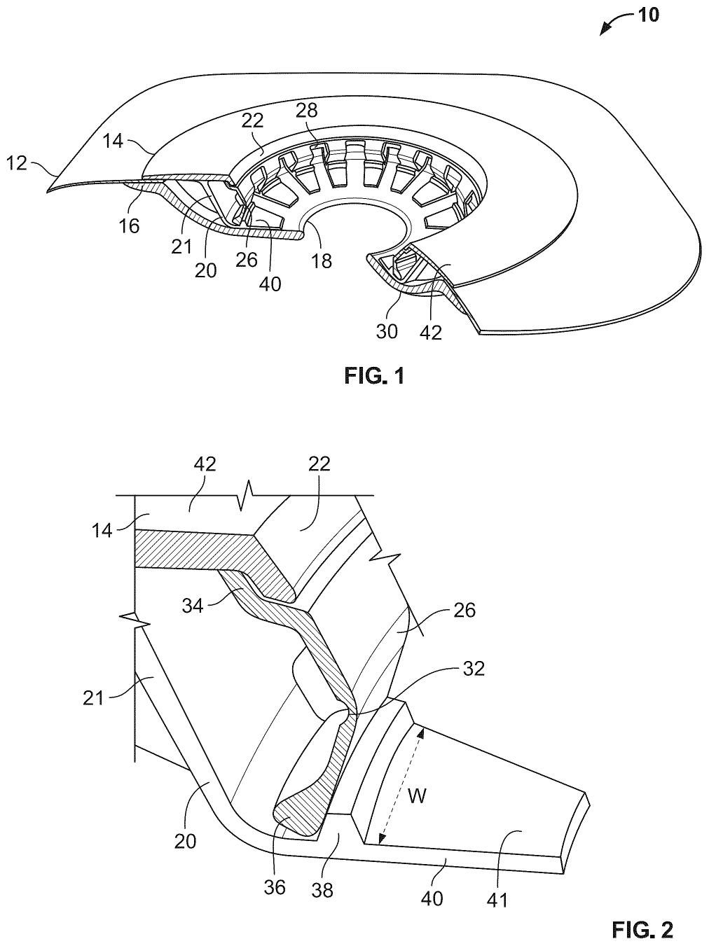

Referring now to the figures, FIGS. 1-6 show an ostomy barrier appliance according to several embodiments. FIG. 1 is a perspective view of the ostomy barrier appliance 10 with a portion removed and viewed from a pouch-facing side (also referred to herein as a distal side) to illustrate a layered construction of the ostomy barrier appliance 10. The ostomy barrier appliance 10 may generally include a tape 12, a skin barrier 16, a convexity adjusting device 14, and an inlet opening 18 for receiving a stoma (not shown). The convexity adjusting device 14 may include a plurality of radially extending members 20 and adjusting members 26, which may be configured to adjust at least one convexity characteristic of the ostomy barrier appliance 10. In an embodiment, the convexity adjusting device 14 may be configured to provide localized adjustment of slope, compressibility and/or depth of the ostomy barrier appliance 10 by manipulating one or more adjusting members 26. The convexity adjusting device 14 may include a base 42 having a lip 22 defined at inner peripheries thereof and may be attached to a distal side of the skin barrier 16 or provided as a convex insert that may be inserted into the ostomy barrier appliance 10.

The tape 12 may include a first adhesive layer and a first backing layer. The first adhesive layer of the tape 12 may be formed from a suitable medical adhesive, such as an acrylic adhesive. The first backing layer may be formed from a suitable material, such as a nonwoven material or a thin polymeric film. In another embodiment, the tape 12 may comprise a hydrocolloid adhesive and a film backing layer.

In some embodiments, the skin barrier 16 may include a second backing layer laminated on the pouch-side surface of the skin barrier 16. The second backing layer may be formed from a suitable heat sealable polymeric material, such that the backing layer may be heat sealed to the tape 12. The skin barrier 16 may be formed from a suitable skin-contact adhesive, such as hydrocolloid adhesives.

In an embodiment, the convexity adjusting device 14 may include a base 42 and a plurality of radially extending members 20 extending from the base 42, wherein each of the plurality of radially extending members 20 may be provided with a separate adjusting member 26. The plurality of radially extending members 20 may include a dome portion 40 and a middle portion 21 having an axial slope and extending between the base 42 and the dome portion 40, such that the base 42 and the dome portion 40 may be arranged in different axial planes to provide a convexity. The plurality of radially extending members 20 and adjusting members 26 may be configured to define and support a convexity of the skin barrier 16. The radially extending members 20 may be attached to the skin barrier 16 at least at the dome portion 40. Each of the plurality of radially extending members 20 may include a tab 38 protruding from a distal surface thereof. An inner peripheral portion 41 of the plurality of radially extending members 20 may be configured to have a width W that is narrower or flush with tab 38.

Each of the plurality of radially extending members 20 may be separated from an adjacent radially extending member 20 by a gap 28, such that each radially extending member 20 may be adjusted relatively independent from each other to allow localized adjustment of at least one characteristic of the convexity of the ostomy barrier appliance 10 to provide a customized barrier appliance according to user's peristomal topography. In an embodiment, the convexity adjusting member 14 may include about 5 to 20 radially extending members 20. For example, the convexity adjusting member 14 may include 16 radially extending members 20. In an embodiment, the ostomy barrier appliance 10 may be configured as a convex ostomy barrier including a dome 30 supported by the plurality of radially extending members 20 and adjusting members 26.

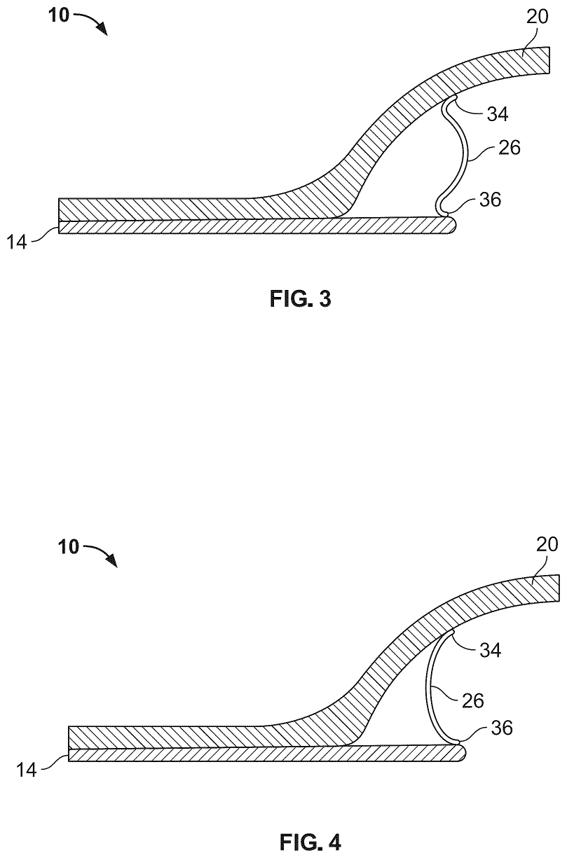

In an embodiment, the adjusting members 26 may be bi-stable clips 26 that are configured to adjust the firmness or compressibility of the radially extending members 20. The bi-stable clips 26 may be configured to be positioned and held in at least two different stable positions, wherein each stable position may provide a different slope, compressibility and/or depth of the corresponding radially extending member 20. In an embodiment, each of the plurality bi-stable clips 26 may be configured to be positioned and held in a first position and a second position, wherein the bi-stable clip 26 may be clipped forward, towards the stoma (as shown in FIG. 3) into the first position to provide a relatively firmer compressibility, more aggressive slope, and greater depth, and pushed backward, away from the stoma (as shown in FIG. 4) into the second position to provide a relatively softer compressibility, less aggressive slope, and lesser depth.

In an embodiment, the ostomy barrier appliance 10 may be provided with each of the bi-stable clips 26 arranged in the first position to provide a convex barrier appliance having a uniform and relatively firm compressibility. In use, a user may manipulate one or more of the plurality of bi-stable clips 26 to change the position of the bi-stable clip 26 to the second position to reduce the compressibility or firmness of the convex barrier at desired locations. For example, a user may manipulate the bi-stable clips 26 arranged at 3 o'clock and 9 o'clock positions of the convex ostomy barrier 10 to the second position to provide a relatively softer compressibility in these areas to better conform to user's skin folds and creases. As such, localized and customized adjustment of the convex ostomy barrier 10 may be achieved by changing the position of one or more of the plurality of bi-stable clips 26. In another embodiment, a user may change the position of the bi-stable clip 26 to the second position to increase the compressibility or firmness of the convex barrier at desired locations.

FIG. 3 shows a bi-stable clip 26 being clipped forward in the first position to provide a relatively firm first compressibility of the convex ostomy barrier 10. FIG. 4 shows a bi-stable clip 26 being clipped backward in the second position to provide a relatively soft second compressibility of the convex ostomy barrier 10, wherein the first compressibility is greater than the second compressibility.

FIG. 2 is a partial enlarged view of the convexity adjusting device 14 of FIG. 1, wherein the bi-stable clips 26 is arranged in the first position. The bi-stable clip 26 may include a first end 34 sealed to an inner surface of the lip 22, a second end 36 arranged against the tab 38, and a hinge 32 defined between the first and second end and configured to allow the bi-stable clip 26 to bend and change between the first position and the second position. In the embodiment of FIGS. 1 and 2, the hinge 32 may be formed by an indentation or score provided on an inner surface of the bi-stable clip 26 facing the corresponding radially extending member 20. In an embodiment, the plurality of bi-stable clips 26 may be formed as a single-piece adjusting device, wherein each of the plurality of bi-stable clips 26 may be attached to and extending from a ring-like base that forms the first end 34 of each of the plurality of bi-stable clip 26 and connects adjacent bi-stable clips 26 as shown in FIG. 1.

The convexity adjusting device 14 may be formed from a suitable material, such as polymeric materials, rubber, silicone, or metallic materials. For example, the convexity adjusting device 14 may be formed from a heat sealable thermoplastic material, such as ethylene vinyl acetate (EVA), thermoplastic elastomer, or thermoplastic urethane.

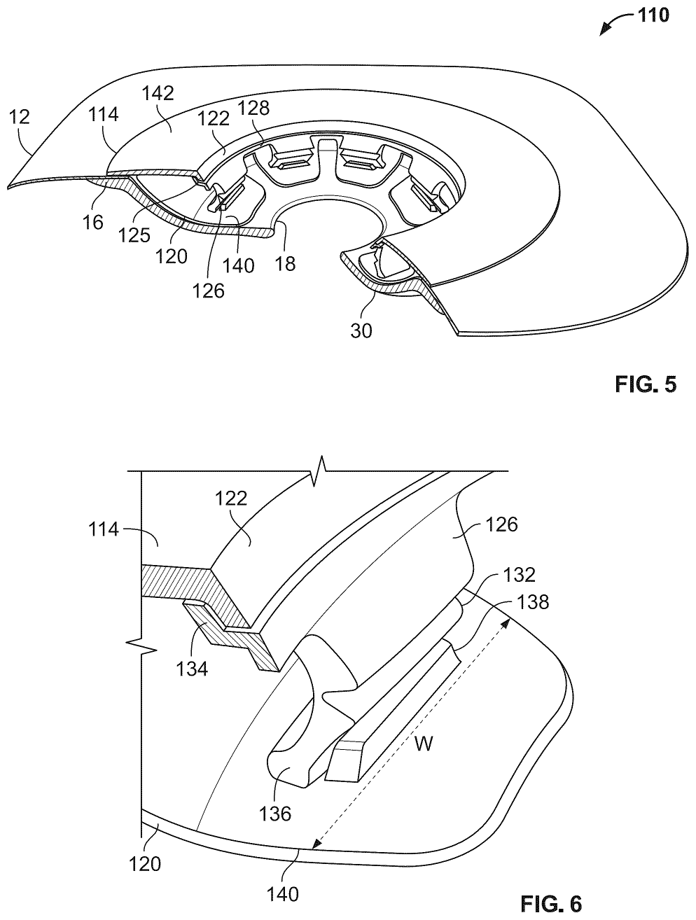

FIGS. 5 and 6 show an ostomy barrier appliance 110 according to another embodiment. The ostomy barrier appliance 110, may be configured similar to ostomy barrier appliance 10 of FIG. 1, and may generally include a tape 12, a skin barrier 16, a convexity adjusting device 114, and an inlet opening 18 for receiving a stoma. The convexity adjusting device 114 may be formed as a convex insert 114 arranged adjacent a distal surface of the skin barrier 16 and comprising a base 142, a plurality of radially extending members 120, and a plurality of adjusting members 126. The plurality of adjusting members 126 may be attached to and extending from an adjusting member base 125 and configured for localized adjustment of at least one convexity characteristic of the ostomy barrier appliance 110.

The radially extending members 120 may be extending from the base 142 and configured to provide a convexity to the skin barrier 16. In an embodiment, the convexity adjusting device 114 may be attached to the tape 12 at the base 142 and attached to the skin barrier 16 at a dome portion 140. The convexity adjusting device 114 may include a lip 122 and a tab 138, wherein the plurality of adjusting members 126 may be secured in place between the lip 122 and the tab 138. In the embodiment of FIGS. 5 and 6, the radially extending member 120 may have a width W that extends past the edges of the tab 138. Each of the plurality of radially extending members 120 may be separated from an adjacent radially extending member 120 by a gap 128.

In an embodiment, the ostomy barrier appliance 110 may have 8 radially extending members 120.

FIG. 6 is a partial enlarged view of the convexity adjusting device 114 of FIG. 5. Similar to the adjusting members 26 of FIGS. 1 and 2, the adjusting members 126 may be formed as bi-stable clips 126 comprising a first end 134 sealed to an inner surface of the lip 122, a second end 136 arranged against the tab 138, and a hinge 132 defined between the first and second end and configured to allow the bi-stable clip 26 to bend and change between a first position and a second position. In this embodiment, the hinge may be formed by an indentation or score provided on an outer surface of the bi-stable clip 126 facing the stoma (not shown). In another embodiment, the score may be formed on the inner surface of the bi-stable clip 126 distal to the stoma (not shown).

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A convex ostomy barrier assembly for attaching an ostomy appliance to a peristomal skin surrounding a stoma, comprising:
a skin barrier comprising an adhesive;
an inlet opening defined in the skin barrier for receiving the stoma; and
a convexity adjusting device, wherein the convexity adjusting device is configured to provide a convexity to the skin barrier, and wherein the convexity adjusting device is configured to provide a localized and customized adjustment of at least one characteristic of the convexity, wherein the at least one characteristic of the convexity includes a compressibility or slope.

2. The convex ostomy barrier assembly of claim 1, wherein the convexity adjusting device comprises:
a plurality of radially extending members, wherein each of the plurality of radially extending members is separated from an adjacent radially extending member by a gap; and
a plurality of adjusting members,
wherein each of the plurality of radially extending members is configured to be adjusted independently by one of the plurality of adjusting members, and wherein each of the plurality of radially extending members and the corresponding adjusting member are configured to provide a localized adjustment of a slope, compressibility and/or depth of the convexity.

3. The convex ostomy barrier assembly of claim 2, wherein the plurality of radially extending members comprise eight radially extending members.

4. The convex ostomy barrier assembly of claim 2, wherein the plurality of radially extending members comprise sixteen radially extending members.

5. The convex ostomy barrier assembly of claim 2, wherein each of the plurality of adjusting members is formed by a bi-stable clip configured to adjust the slope, compressibility and/or depth of the convexity of the skin barrier, wherein the bi-stable clip is configured to be positioned and held in at least two different stable positions, wherein each stable position provides a different slope, compressibility and/or depth of the corresponding radially extending member.

6. The convex ostomy barrier assembly of claim 5, wherein the at least two different stable positions include a first position and a second position, wherein the radially extending member provides a higher compressibility when the corresponding bi-stable clip is set in the first position when compared to the second position.

7. The convex ostomy barrier assembly of claim 2, wherein the convex adjusting device is formed by a convex insert comprising a base including a lip defined at an inner periphery, wherein the plurality of radially extending members extends from the base, wherein each of the radially extending members includes a tab, wherein each of the plurality of adjusting members is secured in place between the lip and the tab.

8. The convex ostomy barrier assembly of claim 7, wherein the bi-stable clip includes a first end, a second end, and a hinge arranged between the first and second end, wherein in the hinge is configured to allow the bi-stable clip to bend and change position between the first position and the second position.

9. The convex ostomy barrier assembly of claim 8, wherein the first end of the bi-stable clip is attached to an inner surface of the lip of the base and the second end of the bi-stable clip is arranged against the tab.

10. The convex ostomy barrier assembly of claim 8, wherein the hinge is formed by an indentation or score provided in the bi-stable clip.

11. The convex ostomy barrier assembly of claim 10, wherein the hinge is defined in an inner surface of the bi-stable clip facing the corresponding radially extending member.

12. The convex ostomy barrier assembly of claim 10, wherein the hinge is defined in an outer surface of the bi-stable clip facing the inlet opening.

13. The convex ostomy barrier assembly of claim 6, wherein the convex ostomy barrier assembly is provided with each of the plurality of bi-stable clips arranged in the first position to provide a generally uniform compressibility of the convexity, wherein the convex ostomy barrier assembly is configured to allow a user to manipulate one or more of the plurality of bi-stable clips to change the position of the bi-stable clips to the second position to adjust the compressibility at one or more locations.

14. The convex ostomy barrier assembly of claim 2, wherein the plurality of adjusting members are attached to an adjusting member base and formed as a single-piece device, wherein the adjusting member base forms a first end of each of the plurality of adjusting members and adjacent adjusting members are connected via the adjusting member base.
